(19) United States
(12) Patent Application Publication
(10) Pub. No.: US 2020/0360403 A1
(43) Pub. Date: Nov. 19, 2020

(54) OPTHALMIC COMPOSITIONS FOR INHIBITING CLOUDING OF THE OCULAR LENS

(71) Applicant: Joseph Domenic SCIAMANNA, Elkton, MD (US)

(72) Inventor: Joseph Domenic SCIAMANNA, Elkton, MD (US)

(21) Appl. No.: 16/713,578

(22) Filed: Dec. 13, 2019

Related U.S. Application Data
(60) Provisional application No. 62/942,474, filed on Dec. 2, 2019.
(60) Provisional application No. 62/848,750, filed on May 16, 2019.

(51) Int. Cl. A61K 31/575; A61K 31/724; A61K 9/00

(52) U.S. Cl. CPC A61K 31/575; A61K 9/0048; A61K 31/724

(57) ABSTRACT

Compositions containing an oxysterol or a precursor thereof and a cyclodextrin and methods for use of these compositions in inhibiting or preventing formation of crystallin proteins in the lens of the eye thereby inhibiting clouding or increasing clarity of the eye lens are provided.

This patent application claims the benefit of priority from U.S. Provisional Application Ser. No. 62/942,474, filed Dec. 2, 2019 and U.S. Provisional Application Ser. No. 62/848,750, filed May 16, 2019, the contents of each of which are herein incorporated by reference in their entireties.

### FIELD

This disclosure relates to compositions containing an oxysterol or a precursor thereof and a cyclodextrin and methods for use of these compositions in inhibiting or preventing formation of crystallin proteins in the lens of the eye thereby inhibiting clouding and/or increasing clarity of the eye lens.

### BACKGROUND

The lens of the eye is an important part of the dioptric system. The lens is a transparent, biconvex structure in the eye that, along with the cornea, helps to refract light to be focused on the retina. Any opacity or clouding of the lens of the eye leads to an overall reduction in eye health and vision.

Traditionally recognized treatment for clouding of the lens which occurs in cataracts is surgery. Cataract surgery often requires the lens of the eye to be removed and replaced with an artificial lens. However, this procedure is both costly and requires recovery time.

The paradigm that cataracts are irreversible and that vision from cataracts can only be restored through surgery has recently been challenged by reports that oxysterols such as lanosterol and 25-hydroxycholesterol can restore vision by binding to αB-crystallin chaperone protein to dissolve or disaggregate lenticular opacities (Daszynski et al. Sci Rep 2019 9:8459 doi:10.1038/s41598-019-44676-4). However, in in vitro rat lens studies and human lens protein solubilization studies, Daszynski et al. showed the compared to ATP, both oxysterols failed to reach the acceptable threshold binding scores for good predictive binding to the cffl-crystallins. All studies by Daszynski et al. failed to provide evidence that lanosterol or 25-hydroxycholesterol have either anti-cataractogenic activity or bind aggregated lens protein to dissolve cataracts (Sci Rep 2019 9:8459 doi:10.1038/s41598-019-44676-4).

There is a need for alternative treatments for ocular lens opacities.

### SUMMARY

An aspect of this disclosure relates to a composition comprising an oxysterol or a precursor thereof and a cyclodextrin.

Another aspect of the present invention relates to a method for inhibiting or preventing formation of crystallin proteins in the lens of the eye. In this method, a composition comprising an oxysterol or a precursor thereof and a cyclodextrin is administered topically to the eye so that formation of crystallin proteins in the lens of the eye is prevented or inhibited.

Another aspect of the present invention relates to a method for inhibiting clouding and/or increasing clarity of the ocular lens. In this method, a composition comprising an oxysterol or a precursor thereof and a cyclodextrin is administered topically to the eye so that clouding of the ocular lens is inhibited and/or clarity of the lens is increased.

Yet another aspect of the present invention relates to a method for treating cataracts in a subject. In this method, a composition comprising an oxysterol or a precursor thereof and a cyclodextrin is administered topically to the eye of a subject suffering from cataracts so that the cataract is treated.

### DETAILED DESCRIPTION

Provided by this disclosure are compositions containing an oxysterol or a precursor thereof and cyclodextrin and methods for their use in inhibiting or preventing formation of crystallin proteins in the lens of the eye thereby inhibiting clouding and/or increasing clarity of the eye lens.

Compositions of this disclosure provide a bio-compatible, aqueous, lipid-soluble molecule delivery system for passage of oxysterols through lipid barriers present in the eye and into the afflicted lens of the eye.

Oxysterols useful in the compositions are capable of reducing the crystallin protein aggregate that causes cataracts. Examples include but are not limited to lanosterol, dihydrolanosterol, 25-hydroxycholesterol, 27-hydroxycholesterol, squalene, and triterpenoids, as well as analogues, prodrugs or precursors thereof. A nonlimiting example of a useful precursor of lanosterol is dihydrolanosterol. In one nonlimiting embodiment, the composition comprises a mixture of both lanosterol and dihydrolanosterol.

In one nonlimiting embodiment, the oxysterol is present in a range of between 0.01%-2% weight by volume of the aqueous composition. Compositions may include an amount of oxysterol ranging from 1 mm to 250 mm concentrations.

Compositions of the present invention further comprise a cyclodextrin. In one nonlimiting embodiment, the cyclodextrin is beta-cyclodextrin or an analogue of beta-cyclodextrin. In one nonlimiting embodiment, the composition comprises beta-cyclodextrin and at least one analogue of beta-cyclodextrin.

In one nonlimiting embodiment, the cyclodextrin is present in a range of between 15%-25% weight by volume of the aqueous composition.

Without being bound to any particular theory, it is believed that the oxysterol molecule acts as a guest in the host cavity of the cyclodextrin. The formulation results in an equilibrium of free oxysterol to complexed oxysterol. The resulting composition allows for the passage of an oxysterol such as lanosterol through lipid barriers present in the eye and into the afflicted lens of the eye. As the aqueous solution is biocompatible with eye tissue, the compositions of this disclosure provide a distinct advantage over compositions containing ethyl alcohols and other non-biocompatible constituents.

The compositions of this disclosure further comprise deionized water and one or more of an emulsifier, a surfactant, a thickening agent, a stabilizer, a penetration enhancer, a salt and/or a lubricant.

Nonlimiting examples of penetration enhancers include mineral oils (petrolatum), various glycols such as, but not limited to propylene glycol, hydroxypropyl guar and ethylene glycol, glycerin and alcohols such as ethanol or benzyl alcohol. In addition, surfactants in the compositions can act as penetration enhancers.

Biocompatible salts or buffers can be used in the compositions to maintain the osmolarity between 50 and 2000 mOsm/kg with a preferable osmolarity being 300 mOsm/kg. In one nonlimiting embodiment, a 0.6% W/V NaCl concentration is used to facilitate complexing interactions and maintain osmolarity.

Examples of emulsifiers which can be used include, but are not limited to, hydroxypropyl-beta-cyclodextrin in addition to the cyclodextrin agent used for complexing, polysorbate-80 and polyacrylic acid, as well as other emulsifiers known in the art.

Nonlimiting examples of thickening agents or stabilizers include, but are not limited to, carboxymethylcellulose, hydroxypropyl methylcellulose and hydroxy ethyl methyl cellulose as well as other similar hygroscopic thickening agents known to the skilled in the art.

For surfactants, a nonionic surfactant is preferred. Nonlimiting examples of commercially available nonionic surfactants include Kolliphor® P 188, Plurionic P123, Lutrol® F68, Poloxamer 188, and poly(ethylene glycol)-block-poly(propylene glycol)-block-poly(ethylene glycol) and similar copolymers consisting of ethylene glycol, propylene glycol, propylene oxide and/or similar hydrocarbon polymers consisting of non-ionic monomers.

Nonlimiting examples of lubricants include glycerin, glycerol, castor oil, carbomer, polysorbate 80, hypromellose, carboxymethylcellulose, sodium hyaluronate, hydroxypropyl-guar, polyvinylpyrrolidone, polyethylene glycol 400 and propylene glycol.

In some nonlimiting embodiments, the compositions further comprise a preservative. In one nonlimiting embodiment, the preservative is included in a range between 0.1-0.8% weight by volume. Nonlimiting examples of preservatives include biocompatible preservatives such as benzalkonium chloride 0.005% as well as benzyl alcohol.

In addition, in some nonlimiting embodiments, the compositions further comprise an antioxidant between 0.1 and 0. 3% weight by volume may be added.

Nonlimiting examples of antioxidants useful in the compositions of this disclosure include N-acetylcarnosine, carotenoids, Vitamin A, Vitamin B, Vitamin C, n-acetylcysteine, erythorbic acid, sodium sulfites, sodium thiosulfate, thioglycerol, dithiothreitol and dithioerythreitol, as well as similar biocompatible antioxidants.

Further, in some nonlimiting embodiments, the compositions comprise a buffering agent included for long-term stability. In these nonlimiting embodiments, the buffering agent is included to maintain a biological pH range of pH 4.5-8.0. A preferred pH for the ophthalmic composition is pH 6.2. Nonlimiting examples of buffering agents include citrate buffers, histidine buffers, Tris-HCl, acetate buffers and some phosphate-based buffers. Phosphate buffered saline (PBS) is not compatible with the inclusion complexes above pH 6.0. PBS has also been identified as an ocular calcification agent. Accordingly, it is preferred that phosphate-based buffers other than PBS be used in the compositions of this disclosure.

Compositions of this disclosure are formulated for administration via topical application to the eye. Preferred is that the compositions be administered in the form of eye drops at least once, more preferably at least twice a day to a subject in need. At each administration, at least two drops are administered to each eye.

Compositions of this disclosure can be administered to inhibit or prevent formation of crystallin proteins in the lens of the eye of a subject. Further, administration of the compositions will inhibit clouding and/or increase clarity of the ocular lens of a subject. Thus, the compositions of this disclosure are useful in treating cataracts and other vision disorders relating to lens clarity in a subject.

Subjects to which the compositions may be administered include any mammal which forms unwanted crystallin proteins in the eye tissue. Examples include, but are in no way limited to, but are not limited to, humans, dogs, cats, horses, rabbits, rodents, cattle, and primates.

In one nonlimiting embodiment, the biocompatible compositions of this disclosure are prepared in two steps.

As a nonlimiting example, the first step may involve the formation of inclusion complexes predominantly of a cyclodextrin such as methyl-beta-cyclodextrin and an oxysterol such as lanosterol, and to a lesser extent a cyclodextrin such as hydroxypropyl-beta-cyclodextrin and an oxysterol such as lanosterol. To form the inclusion complexes, desired amounts of the methyl-beta-cyclodextrin, hydroxypropyl-beta-cyclodextrin and lanosterol are mixed with deionized water and then heated to form the precursor solution. The resultant precursor solution is made of complexed molecules of the oxysterol and cyclodextrin such that the aqueous solubility of the oxysterol molecules is significantly increased. The quantity of hydroxypropyl-beta-cyclodextrin may be added in greater quantity than necessary for complexing to improve complex solubility. Alternatively, the inclusion complexes can be formed by freeze drying or pressurizing a solution of these cyclodextrins and lanosterol after dispersion in water, ethanol or a similar solvent. The resulting complex formations can exist either in solution or as a solid before they are added to the bulk of the solution. While not being bound to any particular theory, it is believed that the molar substitution of hydroxypropyl and methyl groups on hydroxypropyl-beta-cyclodextrin and methyl-beta-cyclodextrin, respectively, affect the solubility and enthalpy of inclusion formation of these molecules. Molar substitution must be sufficiently high for provision of aqueous solubility to each cyclodextrin through the addition of hydrogen bonds. However, the degree of substitution also impacts the stability of inclusion complexes. Higher degrees of substitution polarize the molecules, thereby limiting their interaction with nonpolar molecules such as lanosterol. In the compositions of this disclosure, it is believed that the combination of highly-substituted hydroxypropyl-beta-cyclodextrin and low-substitution methyl-beta-cyclodextrin uniquely improves the solubility of both components without sacrificing the stability of methyl-beta-cyclodextrin/lanosterol inclusions. With regard to formulation, methyl-beta-cyclodextrin is expected to have a low-moderate degree of substitution and hydroxypropyl-beta-cyclodextrin is expected have a high degree of substitution.

After the precursor solution of inclusion complexes is completely solubilized and cooled to ambient temperature, in this nonlimiting example, the precursor solution is then added in the second step to a second solution to form the biocompatible composition. The second solution is comprised of deionized water and one or more of an emulsifier, a surfactant, a thickening agent, a stabilizer, and a lubricant. In some nonlimiting embodiments, an antioxidant is also added to the second solution.

In this nonlimiting example, the resulting biocompatible composition has a hydroxypropyl-beta-cyclodextrin content of between 15-25% weight by volume, a methyl-beta-cyclodextrin/lanosterol inclusion complex content between 0.5-5% weight by volume, and a carboxymethylcellulose content between 0.1 and 1.0% weight by volume. Other optional contents of the biocompatible composition include surfactant between 0.5-2.5% weight by volume, lubricant between 0.1-3% weight by volume, and stabilizer of 0.02% weight by volume.

In another nonlimiting embodiment, the biocompatible composition is made by combining an aqueous solution (precursor solution) of methyl-beta-cyclodextrin inclusion complexes which are added to a second solution of hydroxypropyl-beta-cyclodextrin or other emulsifier, not inclusion complexed), non-ionic surfactant, glycerin, thickening agent, stabilizer, and an antioxidant. If desired a preservative may also be included in the second solution.

In yet another nonlimiting embodiment, an aqueous composition is formed having a precursor compound of lanosterol, hydroxypropyl-3-cyclodextrin, and methyl-β-cyclodextrin (MBCD). In this nonlimiting embodiment, the ratio of MBCD to lanosterol is at least a 1:1 MBCD:lanosterol molar ratio. Optionally, the solution may further comprise a nonionic surfactant, hypromellose USP or glycerin. In this nonlimiting embodiment, hydroxypropyl-β-cyclodextrin is present in a range of 1%-50% weight by volume and methyl-β-cyclodextrin is present in a range of 0.05%-30% weight by volume. When the nonionic surfactant is present, it is typical to have between 0.001%-4% weight by volume. When hypromellose USP is present it is in a range of 0.01%-5% weight by volume. When glycerin is present, it is in a range of 0.01-5% weight by volume.

In yet another nonlimiting embodiment, an aqueous composition is formed having a precursor compound of lanosterol, hydroxypropyl-β-cyclodextrin, methyl-β-cyclodextrin (MBCD), a nonionic surfactant at 1% weight by volume, a lubricant such as glycerin USP at 1% weight by volume, carboxy methyl cellulose USP at 0.3% weight by volume, and a preservative such as benzyl alcohol USP at 0.3% weight by volume. In this nonlimiting embodiment, lanosterol is present in a range of 0.1% to 1.0% by weight by volume, and preferably between 0.15 and 0.35% weight by volume; hydroxypropyl-β-cyclodextrin is present in a range between 1%-50% weight by volume; methyl-β-cyclodextrin is present in a range between 0.1%-30% weight by volume; and a nonionic surfactant is present in a range between 0.1% and 2% weight by volume. In this nonlimiting embodiment, an antioxidant such as N-acetyl carnosine can further be added to the formulation. The N-acetylcarnosine, if added, is present in a range between 0.25% and 3% weight by volume. Hypromellose USP can also be added in a range of 0.01%-5% weight by volume as well as a preservative such as benzalkonium chloride in a range of 0.001% to 0.05% weight by volume.

The following nonlimiting examples are provided to further illustrate the present invention.

### EXAMPLES

### Example 1: Preparation of Composition

An aqueous solution was prepared in two steps. The first step involved preparation of inclusion complexes where 72 g of methyl-beta-cyclodextrin (molar substitution 0.6 or higher), 180 g hydroxypropyl-beta-cyclodextrin and 14 grams of lanosterol were mixed with 240 ml of deionized water in a sealed container. The mixture was heated to 90° C. for 6 hours and pressurized to 1100 Torr. The mixture was cooled to ambient temperature slowly for at least 1 hour and then deionized water was added such that the total volume was 800 ml. The pH was adjusted to pH 6.5 through the titration of HCl.

The 800 ml solution of methyl-beta-cyclodextrin inclusion complexes was then added to a 3.2 L solution including the emulsifier hydroxypropyl-beta-cyclodextrin, the non-ionic surfactant Kolliphor P 188, glycerin as a lubricant, benzyl alcohol as a preservative, carboxymethylcellulose as a thickening agent, polysorbate 80 as a stabilizer, and N-acetylcarnosine as an antioxidant. The resulting solution had the final concentration of 22.0% weight by volume of hydroxypropyl-beta-cyclodextrin, 2.15% weight by volume of methyl-beta-cyclodextrin/lanosterol inclusion complex, 0.1% weight by volume of carboxymethylcellulose, 1% weight by volume of Kolliphor P 188, 1% weight by volume of N-acetylcarnosine, 1% weight by volume of glycerin, 0.3% weight by volume of benzyl alcohol and 0.02% weight by volume of polysorbate.

### Example 2: Administration of Composition

A canine subject with cataracts was administered the composition of Example 1 over a 131 day period. The subject received twice daily administrations of the 25 mm lanosterol solution of at least 2 drops per eye for a total of 4 drops per eye per day. The subject was continuously observed to monitor lens opacity, responsiveness and behavior. Continuous application of the composition to the lens of the subject led to improved light responsiveness, increased lens clarity and increased response to distant stimuli.

What is claimed:

1. A composition comprising an oxysterol or a precursor thereof and one or more cyclodextrins in aqueous solution.

2. The composition of claim 1 wherein the oxysterol is lanosterol.

3. The composition of claim 1 comprising lanosterol and dihydrolanosterol.

4. The composition of claim 1 wherein the oxysterol is present in a range of between 0.01%-2% weight by volume of the composition.

5. The composition of claim 1 wherein the cyclodextrin is a beta-cyclodextrin or an analogue thereof.

6. The composition of claim 1 comprising a mixture of methyl-beta-cyclodextrin and hydroxypropyl-beta-cyclodextrin.

7. The composition of claim 1 further comprising one or more of an emulsifier, a surfactant, a thickening agent, a stabilizer, a lubricant, a penetration enhancer or a salt.

8. The composition of claim 1 formulated for topical administration via drops to an eye.

9. A method for inhibiting or preventing formation of crystallin proteins in an ocular lens of a subject, said method comprising administering the composition of claim 1 to an eye of the subject.

10. The method of claim 9 wherein the composition is administered topically.

11. A method for inhibiting clouding and/or increasing clarity of an ocular lens of a subject, said method comprising administering the composition of claim 1 to an eye of the subject.

12. The method of claim 11 wherein the composition is administered topically.

13. A method for treating cataracts in a subject, said method comprising administering the composition of claim 1 to an eye of the subject.

14. The method of claim 13 wherein the composition is administered topically.
